(19) United States
(12) Patent Application Publication
(10) Pub. No.: US 2018/0037740 A1
(43) Pub. Date: Feb. 8, 2018

(54) PHTHALOCYANINE-BASED COMPLEX COMPOUND

(71) Applicant: Dexerials Corporation, Shinagawa-ku, Tokyo (JP)

(72) Inventors: Ryosuke IWATA, Shinagawa-ku, Tokyo (JP); Yasuhisa ISHII, Utsunomiya-shi, Tochigi (JP); Mikihisa MIZUNO, Sendai-shi, Miyagi (JP)

(73) Assignee: Dexerials Corporation, Shinagawa-ku, Tokyo (JP)

(21) Appl. No.: 15/551,288

(22) PCT Filed: Feb. 9, 2016
(86) PCT No.: PCT/JP2016/000675

§ 371 Date: Aug. 16, 2017

(30) Foreign Application Priority Data
Feb. 20, 2015 (JP) 2015-032138

(51) Int. Cl. C09B 47/067; C09B 47/20; C07F 3/06; C07D 487/22; C07F 1/08; C09B 47/12; C09B 47/24

(52) U.S. Cl. CPC C09B 47/0673; C09B 47/0675; C09B 47/0678; C09B 47/12; C09B 47/20; C09B 47/24; C07D 487/22; C07F 1/08; C07F 3/06

(57) ABSTRACT

A phthalocyanine-based complex compound is represented by general formula (1) shown below,

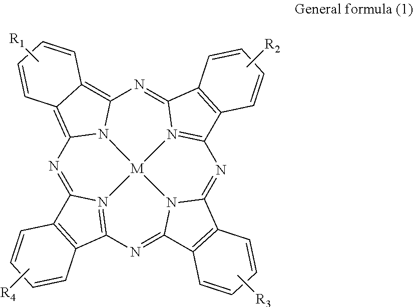

where M in general formula (1) is any of Cu, Fe, Ti, V, Ni, Pd, Pt, Pb, Si, Bi, Cd, La, Tb, Ce, Be, Mg, Co, Ru, Mn, Cr, Mo, Sn, and Zn, and may be present or absent.

### CROSS-REFERENCE TO RELATED APPLICATION

This application claims priority of Japanese Patent Application No. 2015-032138 (filed on Feb. 20, 2015), the entire disclosure of which is incorporated herein by reference.

### TECHNICAL FIELD

The present disclosure relates to a phthalocyanine-based complex compound.

### BACKGROUND

Transparent conductive films that are required to exhibit light transmittivity have been conventionally made from metal oxides such as indium tin oxide (ITO). Examples of such transparent conductive films include a transparent conductive film disposed on a display surface of a display panel such as a touch panel and a transparent conductive film of an information input device disposed at a display surface-side of a display panel. However, a transparent conductive film made using a metal oxide has expensive production costs as a result of being formed by sputtering in a vacuum environment and is susceptible to cracking and delamination due to deformation by bending, warping, or the like.

Consequently, transparent conductive films made using metal nanowires are being considered as an alternative to transparent conductive films made using metal oxides. This is because a transparent conductive film made using metal nanowires can be formed by coating or printing and is highly resistant to bending and warping. Moreover, transparent conductive films made using metal nanowires are attracting attention as next generation transparent conductive films that are made without using the rare metal indium (for example, refer to PTL 1 and 2).

However, the transparent conductive film described in PTL 1 may be tinged red and suffer from loss of transparency.

Moreover, in a situation in which a transparent conductive film made using metal nanowires is disposed at a display surface-side of a display panel, diffuse reflection of external light by the surfaces of the metal nanowires causes black displayed by the display panel to appear slightly brighter, which may be referred to as a “black floating (black level misadjustment)” phenomenon. The black floating phenomenon is a factor that leads to deterioration in display characteristics due to reduced contrast.

A gold nanotube made from gold (Au) has been proposed with the objective of preventing occurrence of the black floating phenomenon since gold has a lower tendency to diffusely reflect light. A gold nanotube is formed by initially using a silver nanowire having a high tendency to diffusely reflect light as a template and subjecting the silver nanowire to gold plating. Thereafter, the silver nanowire portion used as the template is etched or oxidized in order to carry out conversion to a gold nanotube (for example, refer to PTL 3).

Furthermore, a technique for preventing light scattering has been proposed (for example, refer to PTL 2) in which metal nanowires are used in combination with a secondary conductive medium (for example, CNTs (carbon nanotubes), a conductive polymer, or ITO).

However, in the case of the gold nanotube obtained by the former of these methods, not only is the silver nanowire used as a template wasted as a material, but a metal material is also required to perform the gold plating. Therefore, this method suffers from high production costs due to having high material costs and a complicated process.

Furthermore, in the case of the latter of these methods, there may be loss of transparency due to the secondary conductive medium (colorant material), such as CNTs, a conductive polymer, or ITO, being located in openings in a metal nanowire network.

To combat these problems, a transparent conductive film including metal nanowires and a colored compound (dye) adsorbed onto these metal nanowires has been proposed (for example, refer to PTL 4 and 5). In the case of a transparent conductive film including metal nanowires and a colored compound (dye) adsorbed onto the metal nanowires, diffuse reflection of light by the surfaces of the metal nanowires is prevented because the colored compound adsorbed onto the metal nanowires absorbs visible light. Moreover, it is possible to suppress a decrease in transparency of the transparent conductive film caused by addition of the colored compound (dye) because the colored compound (dye), which is for example a colored compound represented by R—X that is composed by a chromophore R and an adsorption functional group X, is adsorbed onto the metal nanowires.

However, mixing of a dye having low conductivity with the metal nanowires may lead to loss of conductivity. Therefore, in a situation in which a dye having low conductivity is used, it is necessary to carry out a pressing process after electrode formation to enhance conductivity, which impairs productivity.

### CITATION LIST

### Patent Literature

PTL 1: JP 2010-507199 A

PTL 2: JP 2010-525526 A

PTL 3: JP 2010-525527 A

PTL 4: JP 2012-190777 A

PTL 5: JP 2012-190780 A

### SUMMARY

### Technical Problem

The present disclosure aims to solve the conventional problems set forth above and achieve the following objective. Specifically, an objective of the present disclosure is to provide a novel phthalocyanine-based complex compound that, when applied onto a metal surface, can improve (i) close adherence to the metal and (ii) durability.

### Solution to Problem

As a result of diligent investigation conducted to achieve the above objective, the inventors discovered that as a result of a specific novel compound including a chromophore and an adsorbate that includes a group having excellent adsorptivity onto metal, when this specific novel compound is used as a dye to surface treat a metal surface, (i) close adherence to the metal and (ii) durability can be improved.

Moreover, as a result of diligent investigation conducted to achieve the above objective, the inventors discovered that as a result of the specific novel compound including a chromophore and an adsorbate that includes a group having excellent adsorptivity onto metal, when this specific novel compound is mixed with a metal filler, (i) affinity to the metal filler, (ii) durability, (iii) inhibition of external light scattering, and (iv) conductivity can be improved. These discoveries led to the present disclosure. All or some of the effects set forth above can also be expected in a situation in which the compound according to the present disclosure is adopted in a metal mesh-type transparent conductive film in which copper, silver, or an alloy based thereon is used.

A constituent element of the metal filler can be selected as appropriate depending on the objective, without any specific limitations other than being a metal element, and may for example be Ag, Au, Ni, Cu, Pd, Pt, Rh, Ir, Ru, Os, Fe, Co, Sn, Al, Tl, Zn, Nb, Ti, In, W, Mo, Cr, V, or Ta. Any one of these metal elements may be used alone or any two or more of these metal elements may be used in combination.

Moreover, the shape of the metal filler can be selected as appropriate depending on the objective, without any specific limitations, and may for example be a spherical shape, a polyhedral shape, a flake shape (thin slice shape), a needle shape, a wire shape, or a prism shape.

The present disclosure is based on the above findings made by the inventors and provides the following as a solution to the problems set forth above. Specifically, the present disclosure provides:

<1> A phthalocyanine-based complex compound represented by general formula (1) shown below,

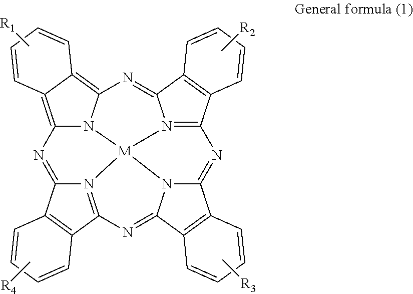

where M in general formula (1) is any of Cu, Fe, Ti, V, Ni, Pd, Pt, Pb, Si, Bi, Cd, La, Tb, Ce, Be, Mg, Co, Ru, Mn, Cr, Mo, Sn, and Zn, and may be present or absent,

one or more of R₁ to R₄ in general formula (1) are present on a phthalocyanine moiety, each include an ion represented by any general formula in general formula group (A) shown below, and may be the same or different to one another,

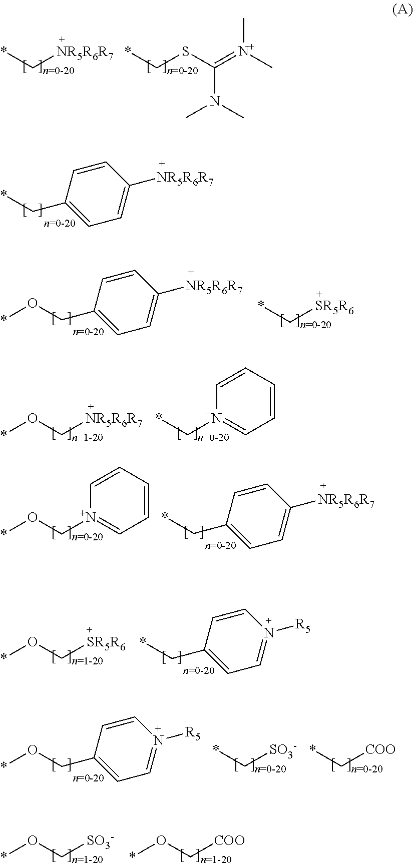

R₅ to R₇ in general formula group (A) are each hydrogen or a hydrocarbon group, and may be the same or different to one another,

R₁ to R₄ in general formula (1) each further include a counter ion represented by any general formula in general formula group (B) shown below,

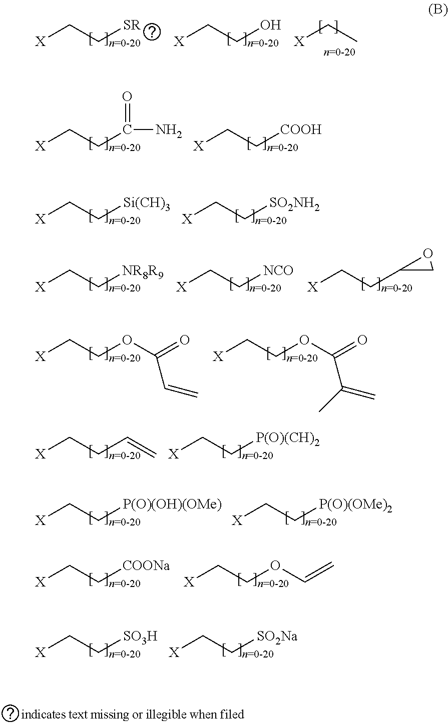

X in general formula group (B) is an ion represented by SO₃⁻, COO⁻, PO₃H⁻, PO₃²⁻, N⁺R₈R₉R₁₀, or PhN⁺R₈R₉R₁₀, an ion represented by general formula (2) shown below, or an ion represented by structural formula (1) shown below,

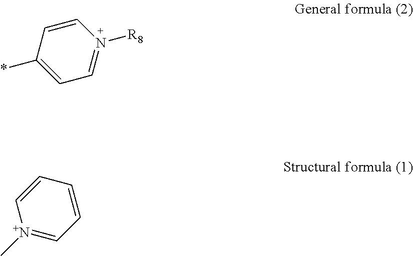

and R₈ to R₁₀ in general formula group (B), N⁺R₈R₉R₁₀, PhN⁺R₈R₉R₁₀, and general formula (2) are each hydrogen or a hydrocarbon group, and may be the same or different to one another.

Note that each of R₁ to R₄ in general formula (1) is bonded to the phthalocyanine moiety at any of a number of specific positions (for example, positions a to d in the following general formula (1) in the case of R₁). The same applies for R₂ to R₄.

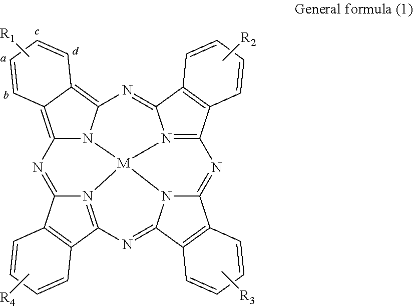

Moreover, the following general formula (X) in general formula group (A) is *—CₙH₂ₙ—N⁺R₅R₆R₇, where n represents an integer of 0 to 20. The same applies for other general formulae in general formula group (A) and general formula group (B). Furthermore, the asterisk (*) in general formula (2), structural formula (1), and the following general formula (X) represents a bonding site to the phthalocyanine moiety.

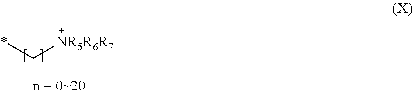

<2> The phthalocyanine-based complex compound according to <1> having an E1% 1 cm value of at least 300 at a wavelength of maximum absorption in a visible light region.

<3> The phthalocyanine-based complex compound according to <1> or <2>, wherein the phthalocyanine-based complex compound dissolves in water or ethylene glycol in an amount of at least 0.01 mass %.

<4> The phthalocyanine-based complex compound according to any one of <1> to <3>, wherein the phthalocyanine-based complex compound dissolves as molecules or disperses as particles having a number average particle diameter of no greater than 3 μm in water or ethylene glycol.

<5> The phthalocyanine-based complex compound according to any one of <1> to <4>, wherein the phthalocyanine-based complex compound forms a solution having a hydrogen ion concentration (pH) of 4 to 10 when dissolved in water in an amount of 0.1 mass %.

<6> A method of producing a phthalocyanine-based complex compound, for use in producing the phthalocyanine-based complex compound according to any one of <1> to <5>, comprising preparing a raw material solution in which a raw material including a phthalocyanine derivative moiety is dissolved in a solvent and a compound solution in which a compound including a moiety that adsorbs onto a metal is dissolved in a solvent, and mixing the raw material solution and the compound solution to precipitate a phthalocyanine-based complex compound.

### Advantageous Effect

According to the present disclosure, it is possible to solve the conventional problems set forth above and achieve the above objective, and to provide a novel phthalocyanine-based complex compound that, when applied onto a metal surface, can improve (i) close adherence to the metal and (ii) durability.

### BRIEF DESCRIPTION OF THE DRAWINGS

In the accompanying drawings:

FIG. 1 schematically illustrates a first embodiment of a transparent electrode containing a phthalocyanine-based complex compound according to the present disclosure;

FIG. 2 schematically illustrates a second embodiment of a transparent electrode containing a phthalocyanine-based complex compound according to the present disclosure;

FIG. 3 schematically illustrates a third embodiment of a transparent electrode containing a phthalocyanine-based complex compound according to the present disclosure;

FIG. 4 schematically illustrates a fourth embodiment of a transparent electrode containing a phthalocyanine-based complex compound according to the present disclosure;

FIG. 5 schematically illustrates a fifth embodiment of a transparent electrode containing a phthalocyanine-based complex compound according to the present disclosure; and

FIG. 6 schematically illustrates a sixth embodiment of a transparent electrode containing a phthalocyanine-based complex compound according to the present disclosure.

### DETAILED DESCRIPTION

### Phthalocyanine-Based Complex Compound

A phthalocyanine-based complex compound according to the present disclosure is represented by general formula (1) shown below.

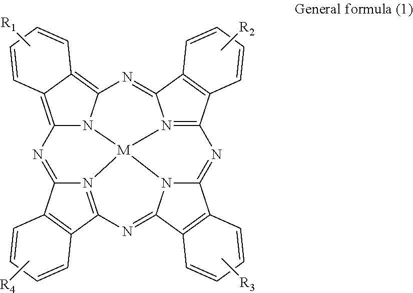

M in general formula (1) is any of Cu, Fe, Ti, V, Ni, Pd, Pt, Pb, Si, Bi, Cd, La, Tb, Ce, Be, Mg, Co, Ru, Mn, Cr, Mo, Sn, and Zn, and may be present or absent.

One or more of R₁ to R₄ in general formula (1) are present on a phthalocyanine moiety, each include an ion represented by any general formula in general formula group (A) shown below, and may be the same or different to one another.

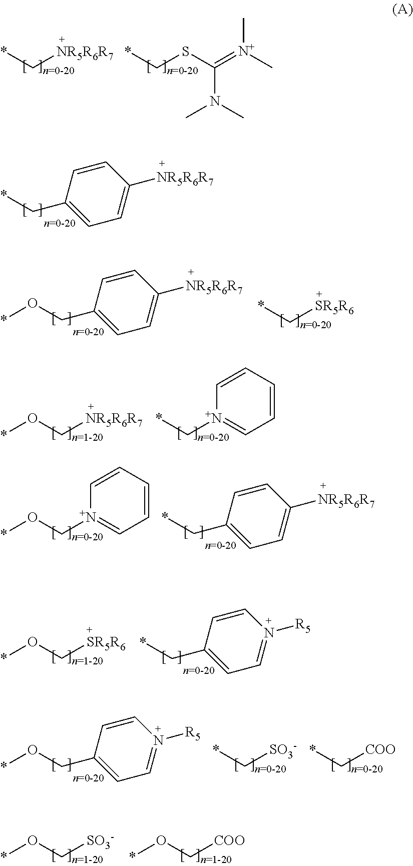

R₅ to R₇ in general formula group (A) are each hydrogen or a hydrocarbon group, and may be the same or different to one another.

R₁ to R₄ in general formula (1) each further include a counter ion represented by any general formula in general formula group (B) shown below.

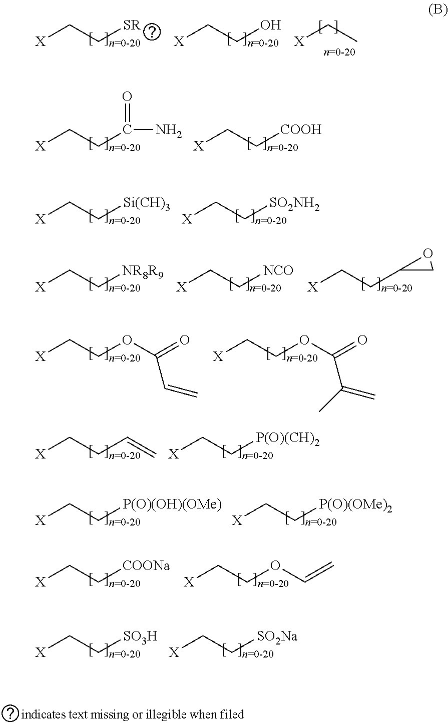

X in general formula group (B) is an ion represented by SO₃⁻, COO⁻, PO₃H⁻, PO₃²⁻, N⁺R₈R₉R₁₀, or PhN⁺R₈R₉R₁₀, an ion represented by general formula (2) shown below, or an ion represented by structural formula (1) shown below.

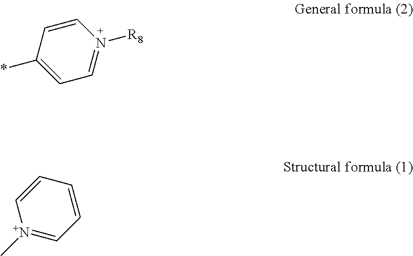

R₈ to R₁₀ in general formula group (B), N⁺R₈R₉R₁₀, PhN⁺R₈R₉R₁₀, and general formula (2) are each hydrogen or a hydrocarbon group, and may be the same or different to one another.

The E1% 1 cm value of the phthalocyanine-based complex compound at a wavelength of maximum absorption in the visible light region can be selected as appropriate depending on the objective, without any specific limitations, and is preferably at least 300, and more preferably at least 400.

When the E1% 1 cm value is at least 300, scattering of external light can be efficiently inhibited, and when the E1% 1 cm value is in the more preferable range, the inhibitive effect on scattering of external light is remarkable.

The solubility of the phthalocyanine-based complex compound with respect to water or ethylene glycol can be selected as appropriate depending on the objective, without any specific limitations, and is preferably at least 0.01 mass % and more preferably at least 0.02 mass % relative to the amount of water or ethylene glycol.

When this solubility is at least 0.01 mass %, the amount of solvent used in surface treatment can be reduced, and when this solubility is in the more preferable range, the amount of solvent can be further reduced, thereby enabling surface treatment to be performed smoothly.

The number average particle diameter of particles of the phthalocyanine-based complex compound when dispersed in water or ethylene glycol can be selected as appropriate depending on the objective, without any specific limitations, and is preferably no greater than 3 μm, and more preferably no greater than 1 μm.

A number average particle diameter of no greater than 3 μm can negate the negative influence of the phthalocyanine-based complex compound on total light transmittivity and a number average particle diameter in the more preferable range can effectively inhibit scattering of external light.

The hydrogen ion concentration (pH) of a solution formed when the phthalocyanine-based complex compound is dissolved in water in an amount of 0.1 mass % can be selected as appropriate depending on the objective, without any specific limitations, and is preferably 4 to 10, and more preferably 5 to 9.

When this hydrogen ion concentration (pH) is 4 to 10, nanowires have a low tendency to be corroded, and when this hydrogen ion concentration (pH) is in the more preferable range, nanowires have a very low tendency to be corroded and have good durability.

<Specific Examples of Phthalocyanine-Based Complex Compound>

The phthalocyanine-based complex compound according to the present disclosure can be selected as appropriate depending on the objective, without any specific limitations, and examples thereof include phthalocyanine-based complex compounds [A] to [H] represented by structural formulae (2) to (9) shown below. Moreover, two or more of such phthalocyanine-based complex compounds may be mixed together.

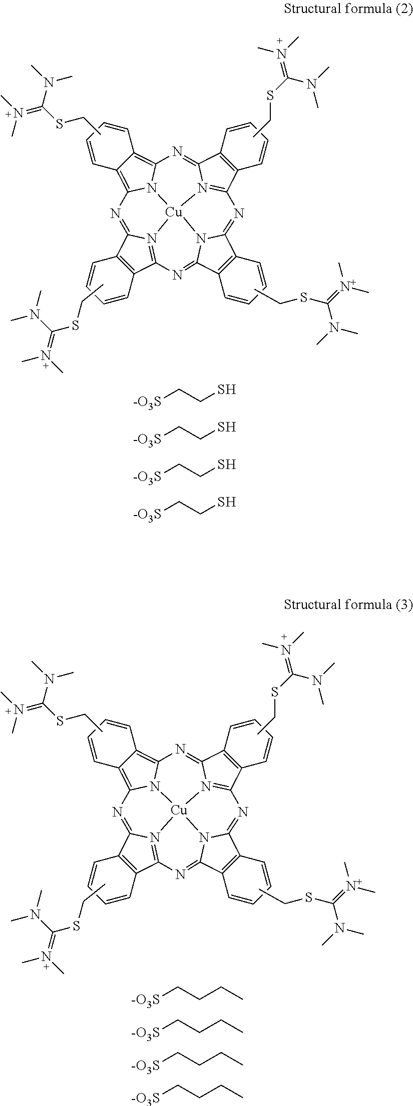

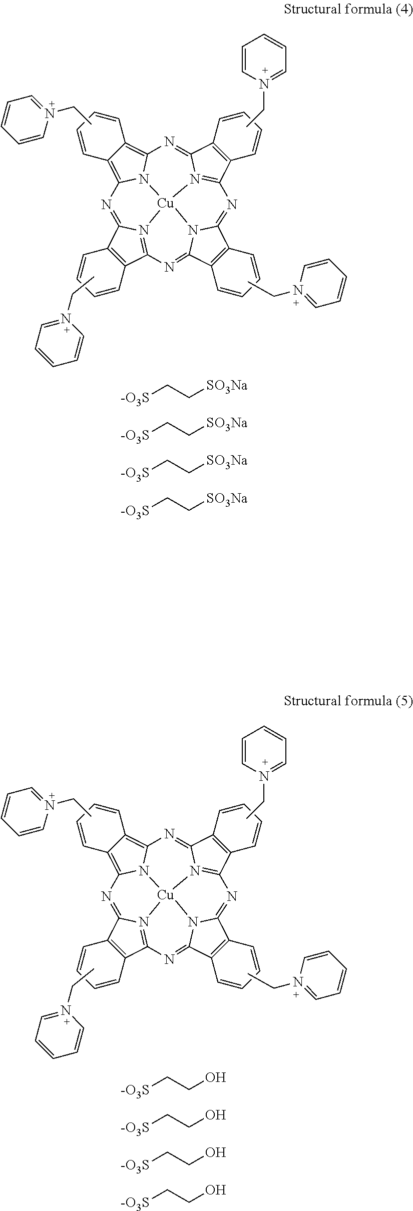

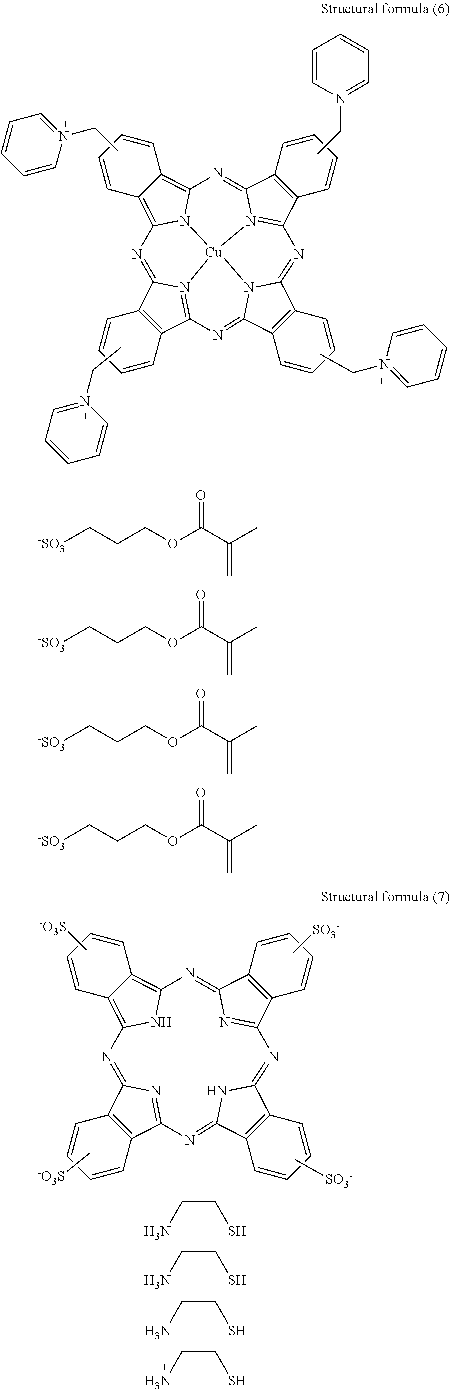

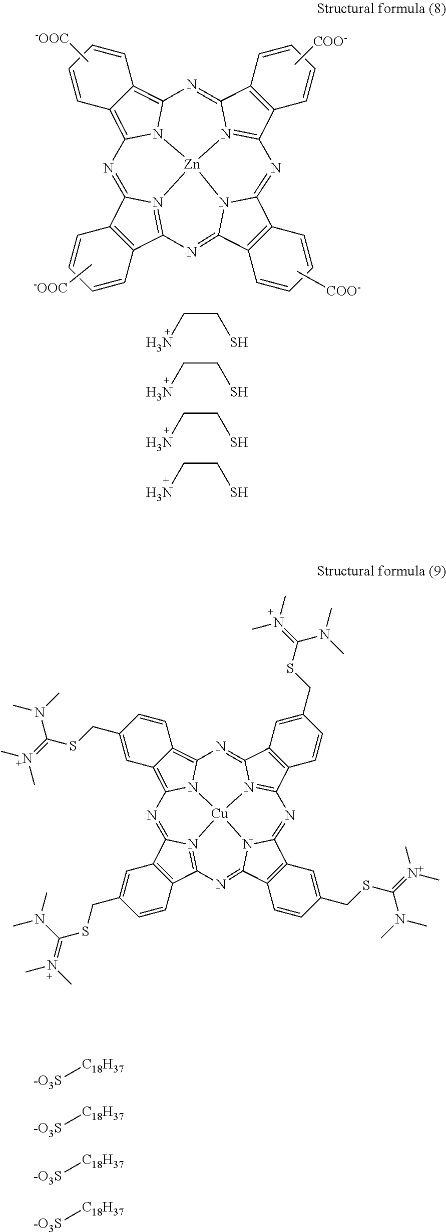

(Method of Producing Phthalocyanine-Based Complex Compound)

The method of producing a phthalocyanine-based complex compound according to the present disclosure includes preparing a raw material solution in which a raw material including a phthalocyanine derivative moiety is dissolved in a solvent and a compound solution in which a compound including a moiety that adsorbs onto a metal (atom group [X] described further below) is dissolved in a solvent, and mixing the raw material solution and the compound solution to precipitate a phthalocyanine-based complex compound. It should be noted that the term “dissolved” as used herein is inclusive of not only a dissolved state but also a dispersed state.

<Raw Material>

The raw material can be selected as appropriate depending on the objective, without any specific limitations, and examples thereof include alcian blue, alcian blue-tetrakis(methylpyridinium) chloride, phthalocyanine tetrasulfonic acid, phthalocyanine monosulfonic acid, phthalocyanine disulfonic acid, phthalocyanine trisulfonic acid, phthalocyanine tetracarboxylic acid, phthalocyanine monocarboxylic acid, phthalocyanine dicarboxylic acid, phthalocyanine tricarboxylic acid, copper phthalocyanine-tetra sulfonic acid tetrasodium salt, copper phthalocyanine-monosulfonic acid tetrasodium salt, copper phthalocyanine-disulfonic acid tetrasodium salt, copper phthalocyanine-tri sulfonic acid tetrasodium salt, copper phthalocyanine-tetracarboxylic acid tetrasodium salt, copper phthalocyanine-monocarboxylic acid tetrasodium salt, copper phthalocyanine-dicarboxylic acid tetrasodium salt, and copper phthalocyanine-tricarboxylic acid tetrasodium salt.

<Compound>

The compound can be selected as appropriate depending on the objective, without any specific limitations, and examples thereof include sodium 2-mercapto-1-ethanesulfonate, sodium butanesulfonate, disodium 1,2-ethanedisulfonate, sodium isethionate, potassium 3-(methacryloyloxy)propanesulfonate, 2-aminoethanethiol, sodium 1-octadecanesulfonate, sodium 3-mercapto-1-propanesulfonate, 2-aminoethanol hydrochloride, sodium 2,3-dimercaptopropanesulfonate, sodium 4-[(5-mercapto-1,3,4-thiadiazol-2-yl)thio]-1-butanesulfonate, sodium mercaptoacetate, sodium 2-(5-mercapto-1H-tetrazol-1-yl)acetate, 5-carboxy-1-pentanethiol sodium salt, 7-carboxy-1-heptanethiol sodium salt, 10-carboxy-1-decanethiol sodium salt, 15-carboxy-1-pentadecanethiol sodium salt, carboxy-EG6-undecanethiol sodium salt, and carboxy-EG6-hexadecanethiol sodium salt.

<Solvent>

The solvent can be selected as appropriate depending on the objective, without any specific limitations, and examples thereof include water; alcohols such as methanol, ethanol, n-propanol, i-propanol, n-butanol, i-butanol, sec-butanol, and tert-butanol; ketones such as cyclohexanone and cyclopentanone; amides such as N,N-dimethylformamide (DMF); and sulfides such as dimethyl sulfoxide (DMSO). The solvent is selected as most appropriate in consideration of raw material and product solubility, and may be a single solvent or a combination of two or more solvents. Furthermore, the solvent may be added partway through. The solution temperature is determined in consideration of raw material and product solubility and the rate of reaction, without any specific limitations.

<Mixing>

The mixing ratio of the raw material solution and the compound solution (raw material solution:compound solution) can be selected as appropriate depending on the objective, without any specific limitations, and is preferably a mass ratio of from 1:0.1 to 1:10.

The mixing time of the raw material solution and the compound solution can be selected as appropriate depending on the objective, without any specific limitations, and is preferably from 1 minute to 48 hours.

<Precipitation>

The precipitation method of the phthalocyanine-based complex compound can be selected as appropriate depending on the objective, without any specific limitations, and examples thereof include a method in which the solution is cooled and a method in which a poor solvent is added.

The following describes a colored compound (dye) formed from the phthalocyanine-based complex compound according to the present disclosure.

<Colored Compound>

The number average particle diameter of the colored compound can be selected as appropriate depending on the objective, without any specific limitations, and is preferably from 0.1 nm to 3 μm, and more preferably from 0.5 nm to 1 μm.

If the number average particle diameter of the colored compound is greater than 3 μm, the colored compound may have a negative effect on total light transmittivity. The number average particle diameter of the colored compound can be measured, for example, using a laser zeta potential meter “ELS-8000” produced by Otsuka Electronics Co., Ltd.

The colored compound preferably absorbs visible region light. Herein, “visible region light” refers to light in a wavelength band from approximately 360 nm or greater to 830 nm or less. The colored compound preferably includes (i) a chromophore that absorbs visible region light and (ii) an atom group including an adsorption group that adsorbs onto a constituent metal of metal nanowires, and is particularly preferably a compound represented by R—X (R is a chromophore that absorbs visible region light and X is an atom group including an adsorption group that adsorbs onto a constituent metal of metal nanowires).

—Chromophore R—

The chromophore R can be selected as appropriate depending on the objective, without any specific limitations other than absorbing visible region light, and may for example be a phthalocyanine derivative.

Among such phthalocyanine derivatives, Cr complexes, Cu complexes, Co complexes, Ni complexes, and Fe complexes are preferable in terms of enabling production of a transparent conductive film having improved transparency.

In the case of phthalocyanine-based complex compounds [A] to [H] represented by the preceding structural formulae (2) to (9), the chromophore R is a phthalocyanine moiety (part excluding R₁ to R₄ in general formula (1)).

—Atom group X—

The atom group X is a moiety including an adsorption group that adsorbs onto a constituent metal of metal nanowires. The atom group X can be selected as appropriate depending on the objective, without any specific limitations other than including the adsorption group, and may for example be a counter ion represented by a general formula in general formula group (B). Specific examples of the adsorption group include a sulfo group (inclusive of sulfonic acid salts), a sulfonyl group, a sulfonamide group, a carboxylic acid group (inclusive of carboxylic acid salts), an aromatic amino group, an amide group, a phosphate group (inclusive of phosphoric acid salts and phosphoric acid esters), a phosphino group, a silanol group, an epoxy group, an isocyanate group, a cyano group, a vinyl group, a thiol group, a sulfide group, a carbinol group, an ammonium group, a pyridinium group, a hydroxy group, and an atom (for example, N (nitrogen), S (sulfur), or O (oxygen)) that can coordinate to a constituent metal of metal nanowires. Any one of these groups may be used alone or any two or more of these groups may be used in combination. Such functional groups are selected as appropriate in consideration of solubility. On the other hand, an alkyl substituted amino group is preferably not used as this group may corrode a metal filler. Herein, “alkyl substituted amino group” refers to an amino group in which all carbon atoms bonded directly to the N atom have sp³ hybridized orbitals. The aforementioned adsorption group is bonded to the chromophore R as the atom group X by covalent bonding or non-covalent bonding. Moreover, the adsorption group may constitute part of the chromophore [R].

Of such adsorption groups, a sulfo group (inclusive of sulfonic acid salts), a thiol group, a carboxylic acid group, and a phosphate group are preferable in terms of suppressing reduction of conductivity due to adsorption of the colored compound.

Examples of the atom group X in the phthalocyanine-based complex compounds [A] to [H] represented by the preceding structural formulae (2) to (9) include counter ions in structural formulae (2) to (9), SO₃⁻ in structural formula (7), and COO⁻ in structural formula (8).

—Method of Producing Colored Compound—

The method by which the colored compound is produced can be selected as appropriate depending on the objective, without any specific limitations. For example, a method may be adopted that involves (I) preparing a solution in which a raw material including a phthalocyanine derivative moiety is dissolved or dispersed in a solvent and a solution in which a compound including a moiety that adsorbs onto a metal is dissolved in a solvent, and (II) mixing the two prepared solutions to precipitate the colored compound.

Examples of the solvent mentioned above include water; alcohols such as methanol, ethanol, n-propanol, i-propanol, n-butanol, i-butanol, sec-butanol, and tert-butanol; ketones such as cyclohexanone and cyclopentanone; amides such as N,N-dimethylformamide (DMF); and sulfides such as dimethyl sulfoxide (DMSO). The solvent is selected as most appropriate in consideration of raw material and product solubility, and may be a single solvent or a combination of two or more solvents. Furthermore, the solvent may be added partway through. The solution temperature is determined in consideration of raw material and product solubility and the rate of reaction, without any specific limitations.

The following describes metal nanowires obtained through adsorption of the phthalocyanine-based complex compound according to the present disclosure onto metal nanowire bodies.

<Metal Nanowires>

The metal nanowires are formed from metal nanowire bodies and the phthalocyanine-based complex compound according to the present disclosure, which is adsorbed onto the metal nanowire bodies as a colored compound.

Adsorption of the colored compound onto the metal nanowire bodies of the metal nanowires can prevent diffuse reflection of light by the surfaces of the metal nanowire bodies because the colored compound absorbs visible light and the like.

Note that the metal nanowires may be inclusive not only of those in which the colored compound is adsorbed onto the entirety of the metal nanowire body, but also those in which the colored compound is adsorbed onto at least part of the metal nanowire body.

<<Metal Nanowire Bodies>>

The metal nanowire bodies are made from metal and are fine wires having nanometer-scale diameters.

A constituent element of the metal nanowire bodies can be selected as appropriate depending on the objective, without any specific limitations other than being a metal element, and may for example be Ag, Au, Ni, Cu, Pd, Pt, Rh, Ir, Ru, Os, Fe, Co, Sn, Al, Tl, Zn, Nb, Ti, In, W, Mo, Cr, V, or Ta. Any one of these examples may be used alone or any two or more of these examples may be used in combination.

Among the constituent elements listed above, Ag or Cu is preferable in terms of having high conductivity.

The average minor axis diameter of the metal nanowire bodies can be selected as appropriate depending on the objective, without any specific limitations, and is preferably from 1 nm to 500 nm, and more preferably from 10 nm to 100 nm.

When the average minor axis diameter of the metal nanowire bodies is less than 1 nm, conductivity of the metal nanowire bodies deteriorates and a transparent conductive film including metal nanowire bodies subjected to adsorption treatment may not be able to function as a conductive film, whereas when the average minor axis diameter of the metal nanowire bodies is greater than 500 nm, a transparent conductive film including metal nanowires obtained through adsorption treatment of the metal nanowire bodies may suffer from poorer total light transmittivity and haze. On the other hand, it is advantageous for the average minor axis diameter of the metal nanowire bodies to be in the preferable range described above because a transparent conductive film including metal nanowires obtained through adsorption treatment of such metal nanowire bodies has high conductivity and high transparency.

The average major axis length of the metal nanowire bodies can be selected as appropriate depending on the objective, without any specific limitations, and is preferably from 1 μm to 100 μm, more preferably from 5 μm to 50 μm and particularly preferably from 20 μm to 50 μm.

When the average major axis length of the metal nanowire bodies is 1 μm or less, the metal nanowire bodies have a poor tendency to join to one another and a transparent conductive film including metal nanowires obtained through adsorption treatment of such metal nanowire bodies may not be able to function as a conductive film, whereas when the average major axis length of the metal nanowire bodies is greater than 100 μm, a transparent conductive film including metal nanowires obtained through adsorption treatment of such metal nanowire bodies may have poor total light transmittivity and haze, and the metal nanowire bodies subjected to adsorption treatment may have poor dispersibility in a dispersion liquid used in formation of the transparent conductive film. On the other hand, it is advantageous for the average major axis length of the metal nanowire bodies to be in the more preferable range or the particularly preferable range described above because a transparent conductive film including metal nanowires obtained through adsorption treatment of such metal nanowire bodies has high conductivity and high transparency.

Note that the average minor axis diameter and the average major axis length of the metal nanowire bodies are respectively a number average minor axis diameter and a number average major axis length that can be measured using a scanning electron microscope. More specifically, at least 100 of the metal nanowire bodies are measured and an image analyzer is used to calculate a projected diameter and a projected area of each nanowire from an electron micrograph. The projected diameter is taken to be the minor axis diameter. The major axis length is calculated based on the following formula.

Major axis length=Projected area/Projected diameter

The average minor axis diameter is the arithmetic mean of the minor axis diameters. The average major axis length is the arithmetic mean of the major axis lengths.

Furthermore, the metal nanowire bodies may alternatively have a wire shape connecting metal nanoparticles in a bead-string shape. No specific limitations are placed on the length in such a situation.

The mass per unit area of the metal nanowire bodies can be selected as appropriate depending on the objective, without any specific limitations, and is preferably from 0.001 g/m² to 1.000 g/m², and more preferably from 0.003 g/m² to 0.03 g/m².

When the mass per unit area of the metal nanowire bodies is less than 0.001 g/m², a transparent conductive film may have poor conductivity because the metal nanowire bodies are not sufficiently present in a metal nanowire layer, whereas when the mass per unit area is greater than 1.000 g/m², a transparent conductive film may have poor total light transmittivity and haze. On the hand, it is advantageous for the mass per unit area of the metal nanowire bodies to be in the more preferable range or the particularly preferable range described above because a transparent conductive film has high conductivity and high transparency in such a situation.

—Method of Producing Metal Nanowires—

The metal nanowires are obtained by mixing the metal nanowire bodies, the colored compound, and a solvent, and also a binder and a dispersant as necessary. For example, the metal nanowires may be obtained by mixing a solid-liquid mixture containing the metal nanowire bodies and the colored compound with a solvent, a binder, and a dispersant, and subsequently stirring the resultant mixture at 20° C. for from 1 minute to 48 hours while performing treatment (surface treatment) to adsorb the colored compound onto the metal nanowire bodies. After the surface treatment, an operation may be carried out to remove colored compound that has not been adsorbed through use of centrifugal separation, filtration, or the like.

The following describes a transparent conductive film including the metal nanowires described above.

<Transparent Conductive Film>

The transparent conductive film includes at least the metal nanowires described above, and may further include other components such as a binder as necessary.

<<Binder>>

The binder is used in order to disperse the metal nanowires and/or metal nanowire bodies, and may be used in the subsequently described dispersion liquid as appropriate.

The binder can be selected as appropriate depending on the objective, without any specific limitations, and examples thereof include known natural polymer resins and synthetic polymer resins that are transparent. The binder may be a thermoplastic resin or a heat (light) curable resin that is cured by heat, light, electron beams, or radiation. Any one of these binders may be used alone or any two or more of these binders may be used in combination.

The thermoplastic resin can be selected as appropriate depending on the objective, without any specific limitations, and may for example be polyvinyl chloride, a vinyl chloride-vinyl acetate copolymer, polymethyl methacrylate, nitrocellulose, chlorinated polyethylene, chlorinated polypropylene, polyvinylidene fluoride, ethylcellulose, hydroxypropyl methylcellulose, polyvinyl alcohol, or polyvinyl pyrrolidone.

The heat (light) curable resin can be selected as appropriate depending on the objective, without any specific limitations, and may for example be melamine acrylate, urethane acrylate, isocyanate, an epoxy resin, a polyimide resin, a silicone resin such as acrylic-modified silicate, or a polymer having a photosensitive group such as an azide group or a diazirine group introduced into either or both of a main chain and a side chain thereof.

<Transparent Electrode Including Transparent Conductive Film>

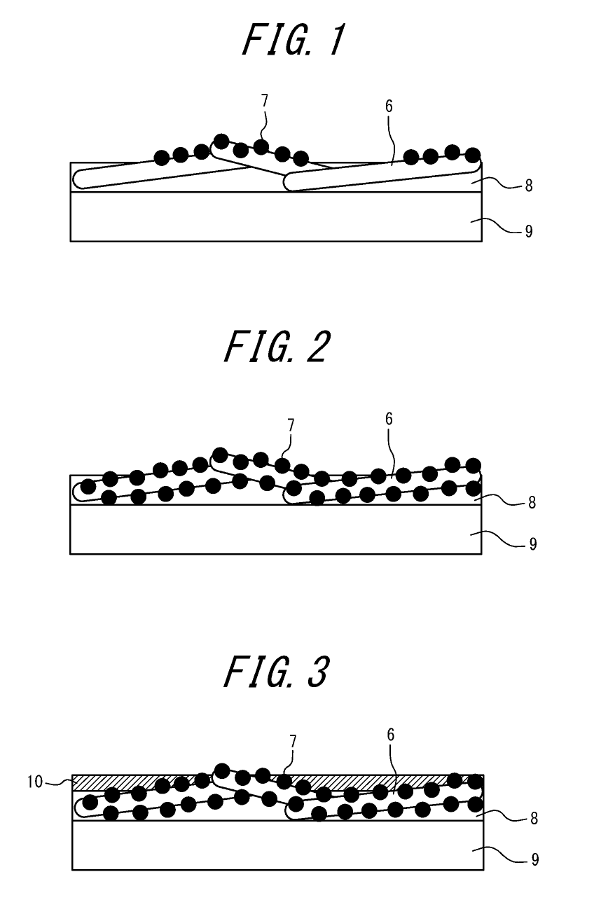

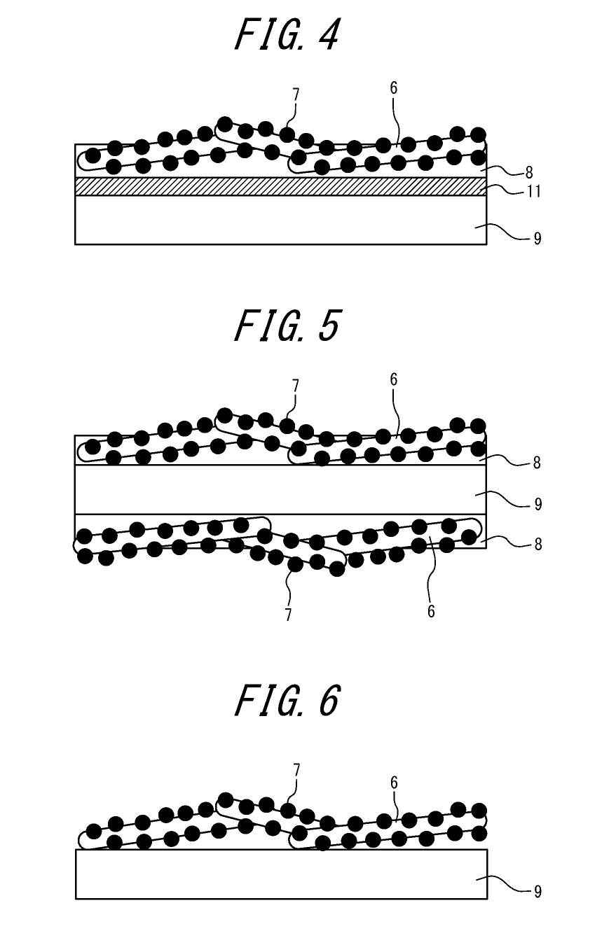

A transparent electrode including the transparent conductive film can be selected as appropriate depending on the objective, without any specific limitations, and may for example be (i) a transparent electrode such as illustrated in FIG. 1 in which a colored compound (dye) 7 is only adsorbed onto sections of metal nanowire bodies 6 that are exposed from a binder layer (the colored compound (dye) 7 may be adsorbed onto the metal nanowire bodies 6, and may be present on part of the surface of the binder layer 8 or within the binder layer 8), (ii) a transparent electrode such as illustrated in FIG. 2 in which a binder layer 8 is formed on a substrate 9 and in which metal nanowires 6 having a colored compound 7 adsorbed thereon are dispersed in the binder layer 8, (iii) a transparent electrode such as illustrated in FIG. 3 in which an overcoating layer 10 is formed on a binder layer 8, (iv) a transparent electrode such as illustrated in FIG. 4 in which an anchor layer 11 is formed between a binder layer 8 and a substrate 9, (v) a transparent electrode such as illustrated in FIG. 5 in which a binder layer 8 including metal nanowire bodies 6 having a colored compound 7 adsorbed thereon is formed on both surfaces of a substrate 9, (vi) a transparent electrode such as illustrated in FIG. 6 in which metal nanowire bodies 6 having a colored compound 7 adsorbed thereon (i.e., metal nanowires) are accumulated on top of a substrate 9 without the colored compound 7 being dispersed in a binder, or (vii) a transparent electrode that is an appropriate combination of any of (i) to (vi).

<<Substrate>>

The substrate can be selected as appropriate depending on the objective, without any specific limitations, and is preferably a transparent substrate made from a material that transmits visible light such as an inorganic material or a plastic material. The transparent substrate is of a thickness required for a transparent electrode including a transparent conductive film, and is for example a film shape (sheet shape) that is thin enough to exhibit flexible bending or a base plate shape that is thick enough to enable an appropriate degree of both bending and rigidity.

The inorganic material can be selected as appropriate depending on the objective, without any specific limitations, and may for example be quartz, sapphire, or glass.

The plastic material can be selected as appropriate depending on the objective, without any specific limitations, and may for example be a commonly known polymer material such as triacetyl cellulose (TAC), polyester (TPEE), polyethylene terephthalate (PET), polyethylene naphthalate (PEN), polyimide (PI), polyamide (PA), an aramid, polyethylene (PE), polyacrylate, polyether sulfone, polysulfone, polypropylene (PP), diacetyl cellulose, polyvinyl chloride, an acrylic resin (PMMA), polycarbonate (PC), an epoxy resin, a urea resin, a urethane resin, a melamine resin, or a cycloolefin polymer (COP). In a situation in which the transparent substrate is made from the plastic material, the thickness of the transparent substrate is preferably from 5 μm to 500 μm from a viewpoint of producibility, but is not specifically limited to this range.

<<Overcoating Layer>>

It is important that the overcoating layer described above displays light transmittivity with respect to visible light. The overcoating layer may for example be made from a polyacrylic-based resin, a polyamide-based resin, a polyester-based resin, or a cellulosic resin, or may be made from a product of hydrolysis and dehydration condensation of a metal alkoxide. The overcoating layer described above is of a thickness that does not impair light transmittivity with respect to visible light. The overcoating layer may have one or more functions selected from the group of functions consisting of hard coating, glare prevention, reflection prevention, Newton ring prevention, and blocking prevention.

<<Anchor Layer>>

The anchor layer described above can be selected as appropriate depending on the objective, without any specific limitations other than being a layer that enables stronger adhesion between the substrate and the binder layer.

<<Method of Producing Transparent Conductive Film>>

The following describes an embodiment of a method of producing the transparent conductive film described above.

The method of producing the transparent conductive film may for example be a method including a dispersion film formation step, a curing step, a calendering step, an overcoating layer formation step, a pattern electrode formation step, and so forth.

<<Dispersion Film Formation Step>>

The dispersion film formation step is a step in which a dispersion film is formed on a substrate using (i) a dispersion liquid containing the metal nanowires described above (i.e., a dispersion liquid in which a colored compound is adsorbed onto metal nanowire bodies) or (ii) a dispersion liquid containing a colored compound, metal nanowire bodies, a binder, and a solvent (i.e., a dispersion liquid in which a colored compound is not adsorbed onto metal nanowire bodies).

Note that the metal nanowires and production method thereof, the colored compound and production method thereof, the metal nanowire bodies, and the binder are the same as previously described, and the solvent is as described further below.

The method by which the dispersion film is formed can be selected as appropriate depending on the objective, without any specific limitations, and is preferably a wet film formation method in terms of physical properties, convenience, production costs, and so forth.

The wet film formation method can be selected as appropriate depending on the objective, without any specific limitations, and may for example be a commonly known method such as a coating method, a spraying method, or a printing method.

The coating method can be selected as appropriate depending on the objective, without any specific limitations, and may for example be micro gravure coating, wire bar coating, direct gravure coating, die coating, dipping, spray coating, reverse roll coating, curtain coating, comma coating, knife coating, or spin coating.

The spraying method can be selected as appropriate depending on the objective, without any specific limitations.

The printing method can be selected as appropriate depending on the objective, without any specific limitations, and may for example be relief printing, offset printing, gravure printing, intaglio printing, rubber plate printing, screen printing, or inkjet printing.

—Dispersion Liquid—

The dispersion liquid contains at least metal nanowires, and may further contain a binder, a solvent, a dispersant, and other additives as necessary. As described above, the metal nanowires include metal nanowire bodies, and are obtained through adsorption of the above-described colored compound onto the metal nanowire bodies.

The metal nanowires and production method thereof, the metal nanowire bodies, and the colored compound and production method thereof are the same as previously described.

The method by which the dispersion liquid is dispersed can be selected as appropriate depending on the objective, without any specific limitations, and suitable examples thereof include stirring, ultrasonic dispersion, bead dispersion, kneading, homogenizer treatment, and pressurized dispersion treatment.

The amount of the metal nanowire bodies of the metal nanowires in the dispersion liquid can be selected as appropriate depending on the objective, without any specific limitations, and is preferably from 0.01 parts by mass to 10.00 parts by mass per 100 parts by mass of the dispersion liquid.

When the amount of the metal nanowire bodies of the metal nanowires is less than 0.01 parts by mass, the metal nanowire bodies do not have a sufficient mass per unit area (0.001 g/m² to 1.000 g/m²) in the finally obtained transparent conductive film, whereas when the amount thereof is greater than 10.00 parts by mass, dispersibility of the metal nanowires deteriorates.

—Binder—

The binder is the same as previously described.

—Solvent—

The solvent can be selected as appropriate depending on the objective, without any specific limitations so long as the solvent allows the number average particle diameter of the colored compound to be maintained at from 0.03 μm to 0.5 μm and enables dispersion of the metal nanowires and/or metal nanowire bodies. Examples of the solvent include water; alcohols such as methanol, ethanol, n-propanol, i-propanol, n-butanol, i-butanol, sec-butanol, and tert-butanol; ketones such as cyclohexanone and cyclopentanone; amides such as N,N-dimethylformamide (DMF); and sulfides such as dimethyl sulfoxide (DMSO). Any one of these solvents may be used alone or any two or more of these solvents may be used in combination.

Of these solvents, an aqueous solvent is preferable in terms of inhibiting scattering of external light. The term “aqueous solvent” refers to a solvent that includes at least water.

—Dispersant—

The dispersant can be selected as appropriate depending on the objective, without any specific limitations, and examples thereof include polyvinyl pyrrolidone (PVP); amino group-containing compounds such as polyethylenimine; and compounds that can be adsorbed onto metal and that have a functional group such as a sulfo group (inclusive of sulfonic acid salts), a sulfonyl group, a sulfonamide group, a carboxylic acid group (inclusive of carboxylic acid salts), an amide group, a phosphate group (inclusive of phosphoric acid salts and phosphoric acid esters), a phosphino group, a silanol group, an epoxy group, an isocyanate group, a cyano group, a vinyl group, a thiol group, or a carbinol group. Any one of these dispersants may be used alone or any two or more of these dispersants may be used in combination.

The dispersant may be adsorbed onto the surfaces of the metal nanowires or metal nanowire bodies. This can improve dispersibility of the metal nanowires or metal nanowire bodies.

In a situation in which the dispersant is added to the dispersion liquid, the additive amount of the dispersant is preferably of a level that does not cause deterioration of conductivity of the finally obtained transparent conductive film. As a result, the dispersant can be adsorbed onto the metal nanowires or metal nanowire bodies in an amount that does not cause deterioration of conductivity of the transparent conductive film.

—Other Additives—

The other additives can be selected as appropriate depending on the objective, without any specific limitations, and examples thereof include thickeners and surfactants.

<<Curing Step>>

The curing step is a step in which the dispersion film formed on the substrate is cured to obtain a cured product. Note that in FIGS. 1-5, the cured product is the binder layer 8 that includes the metal nanowire bodies 6 having the colored compound 7 adsorbed onto the surfaces thereof.

In the curing step, solvent in the dispersion film formed on the substrate is first removed by drying. Removal of the solvent by drying may be carried out by natural drying or heated drying. After the drying, curing treatment of the uncured binder is carried out such that the metal nanowires are in a dispersed state in the cured binder. The curing treatment can be carried out by heating and/or irradiation with activating energy rays.

<<Calendering Step>>

The calendering step is a step that is carried out in order to improve surface smoothness and impart glossiness on the surface.

The calendering can also reduce the sheet resistance value of the obtained transparent conductive film.

<<Overcoating Layer Formation Step>>

The overcoating layer formation step is a step in which an overcoating layer is formed on the cured product that has been formed from the dispersion film.

The overcoating layer can for example be formed by applying, onto the cured product, a coating liquid for overcoating layer formation containing a specific material and curing the applied coating liquid.

<<Pattern Electrode Formation Step>>

The pattern electrode formation step is a step in which a pattern electrode is formed by a commonly known photolithographic process after the transparent conductive film has been formed on the substrate. Through this step, the transparent conductive film according to the present disclosure can be adopted in a sensor electrode for a capacitive touch panel. Furthermore, in a situation in which the curing treatment in the curing step includes irradiation with activating energy rays, the curing treatment may be used for mask exposure/development in formation of the pattern electrode. Also, patterning may be performed by laser etching.

### EXAMPLES

The following provides a more specific description of the present disclosure through examples and comparative examples. However, the present disclosure is not limited to the following examples.

Note that analysis of compounds can be carried out by MALDI-TOF-MS analysis (AXIMA-CFR Plus produced by Shimadzu Corporation) or the like. From the obtained results, it was possible to confirm that the target compound was obtained in each of Examples 1 to 8 described below. Note that “E1% 1 cm” refers to a value at the wavelength of maximum absorption in the visible light region. The E1% 1 cm value was determined with respect to a dye dissolved in DMSO solvent using a V-560 spectrometer produced by JASCO Corporation. The solubility of a phthalocyanine derivative in water was determined by adding 0.01 g of the compound to 10 g of water, using an ultrasonic cleaner for 60 minutes to dissolve the compound and prepare an aqueous solution, subsequently filtering the prepared solution through a PTFE filter having a pore diameter of 3 μm, heating and drying the resultant aqueous solution for 2 hours at 150° C., and then measuring the weight of the residue. The pH was determined through measurement of an aqueous solution prepared in the same manner using a pH meter “F-71S” produced by Horiba, Ltd.

### Example 1

Alcian blue 8GX (produced by Sigma-Aldrich Co. LLC.) and sodium 2-mercapto-1-ethanesulfonate (produced by Wako Pure Chemical Industries, Ltd.) were mixed with a mass ratio of 1:2 in an aqueous medium to prepare a mixed solution. The prepared mixed solution was caused to react for 60 minutes using an ultrasonic cleaner and the resultant reaction liquid was filtered through a 3 μm PTFE filter. Filtered-off solid was washed three times with water and was then dried under reduced pressure to yield a phthalocyanine derivative [A] represented by the following structural formula (2) as a colored compound (dye).

### <MS Analysis Results>

M/Z=289 (phthalocyanine ion moiety)

M/Z=141 (2-mercapto-1-ethanesulfonate ion moiety)

E1% 1 cm: 581

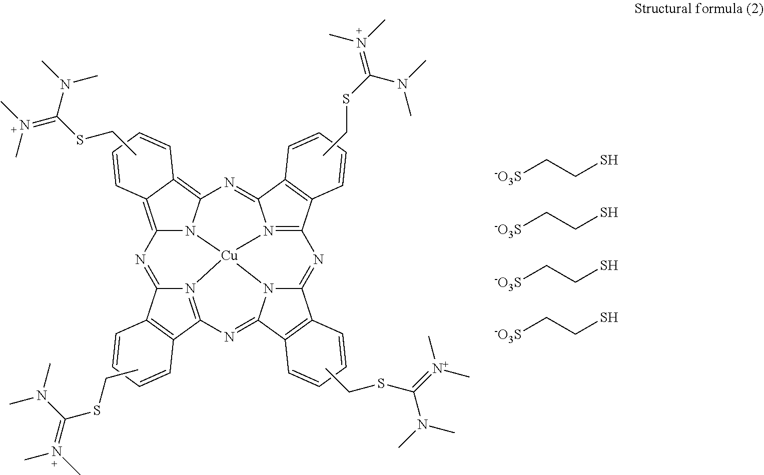

### Example 2

Alcian blue 8GX (produced by Sigma-Aldrich Co. LLC.) and sodium butanesulfonate (produced by Tokyo Chemical Industry Co., Ltd.) were mixed with a mass ratio of 1:2 in an aqueous medium to prepare a mixed solution. The prepared mixed solution was caused to react for 60 minutes using an ultrasonic cleaner and the resultant reaction liquid was filtered through a 3 μm PTFE filter. Filtered-off solid was washed three times with water and was then dried under reduced pressure to yield a phthalocyanine derivative [B] represented by the following structural formula (3) as a colored compound (dye).

### <MS Analysis Results>

M/Z=289 (phthalocyanine ion moiety)

M/Z=137 (butanesulfonate ion moiety)

E1% 1 cm: 578

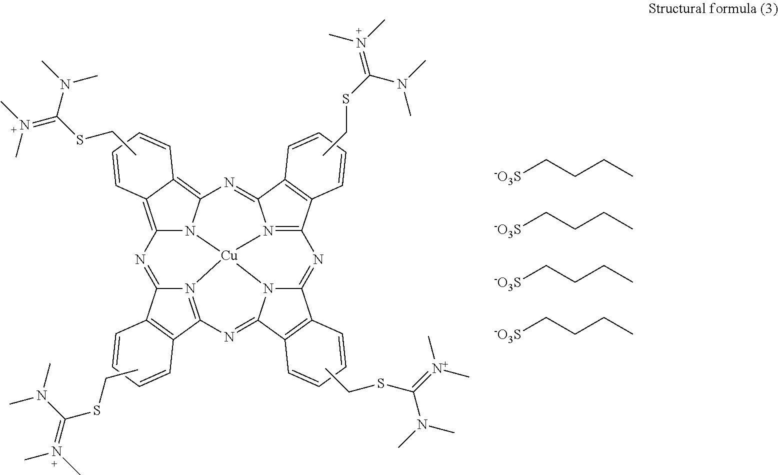

### Example 3

Alcian blue-tetrakis(methylpyridinium) chloride (produced by Sigma-Aldrich Co. LLC.) and disodium 1,2-ethanedisulfonate (produced by Tokyo Chemical Industry Co., Ltd.) were mixed with a mass ratio of 1:2 in methanol to prepare a mixed solution. The prepared mixed solution was caused to react for 60 minutes using an ultrasonic cleaner and the resultant reaction liquid was filtered through a 3 μm PTFE filter. Filtered-off solid was washed three times with methanol and was then dried under reduced pressure to yield a phthalocyanine derivative [C] represented by the following structural formula (4) as a colored compound (dye).

### <MS Analysis Results>

M/Z=236 (phthalocyanine ion moiety)

M/Z=94 (1,2-ethanedisulfonate ion moiety)

E1% 1 cm: 549

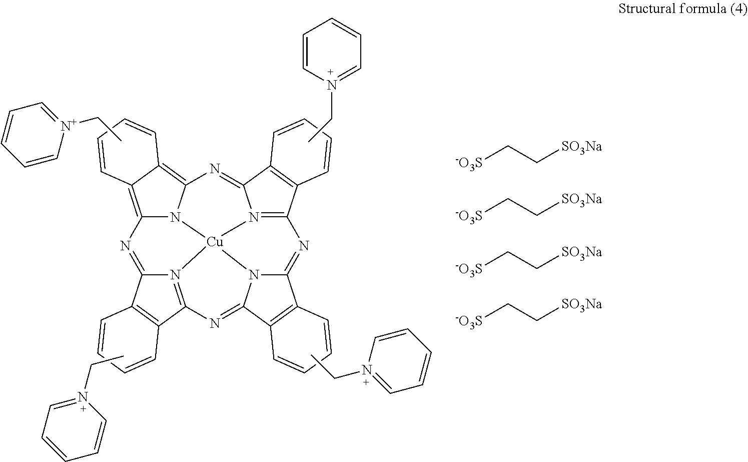

### Example 4

Alcian blue-tetrakis(methylpyridinium) chloride (produced by Sigma-Aldrich Co. LLC.) and sodium isethionate (produced by Tokyo Chemical Industry Co., Ltd.) were mixed with a mass ratio of 1:2 in methanol to prepare a mixed solution. The prepared mixed solution was caused to react for 60 minutes using an ultrasonic cleaner and the resultant reaction liquid was filtered through a 3 μm PTFE filter. Filtered-off solid was washed three times with methanol and was then dried under reduced pressure to yield a phthalocyanine derivative [D] represented by the following structural formula (5) as a colored compound (dye).

### <MS Analysis Results>

M/Z=236 (phthalocyanine ion moiety)

M/Z=125 (isethionate ion moiety)

E1% 1 cm: 692

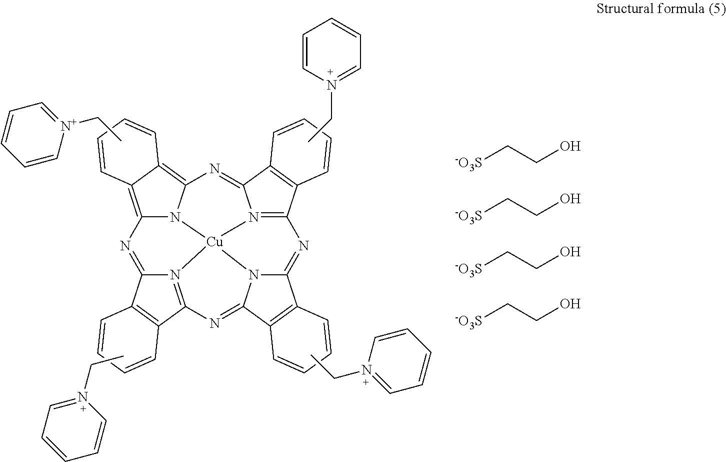

### Example 5

Alcian blue-tetrakis(methylpyridinium) chloride (produced by Sigma-Aldrich Co. LLC.) and potassium 3-(methacryloyloxy)propanesulfonate (produced by Tokyo Chemical Industry Co., Ltd.) were mixed with a mass ratio of 1:2 in methanol to prepare a mixed solution. The prepared mixed solution was caused to react for 60 minutes using an ultrasonic cleaner and the resultant reaction liquid was filtered through a 3 μm PTFE filter. Filtered-off solid was washed three times with methanol and was then dried under reduced pressure to yield a phthalocyanine derivative [E] represented by the following structural formula (6) as a colored compound (dye).

### <MS Analysis Results>

M/Z=236 (phthalocyanine ion moiety)

M/Z=207 (3-(methacryloyloxy)propanesulfonate ion moiety)

E1% 1 cm: 564

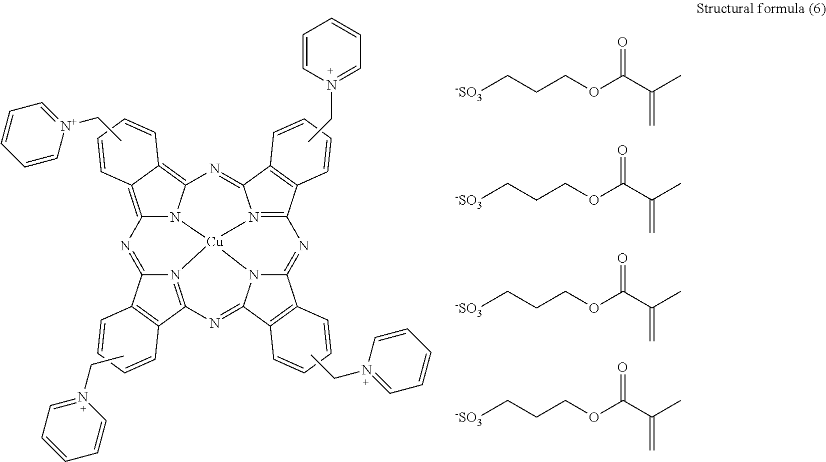

### Example 6

Phthalocyanine tetrasulfonate hydrate (produced by Sigma-Aldrich Co. LLC.) and 2-aminoethanethiol (produced by Tokyo Chemical Industry Co., Ltd.) were mixed with a mass ratio of 1:2 in an aqueous medium to prepare a mixed solution. The prepared mixed solution was caused to react for 60 minutes using an ultrasonic cleaner and the resultant reaction liquid was filtered through a 3 μm PTFE filter. Filtered-off solid was washed three times with water and was then dried under reduced pressure to yield a phthalocyanine derivative [F] represented by the following structural formula (7) as a colored compound (dye).

### <MS Analysis Results>

M/Z=207 (phthalocyanine ion moiety)

M/Z=78 (2-aminoethanethiol ion moiety)

E1% 1 cm: 875

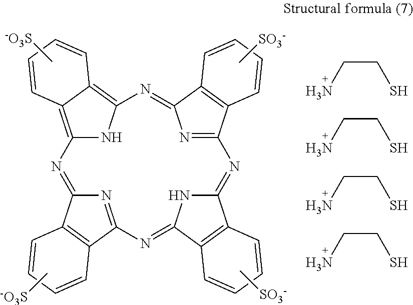

### Example 7

Trimellitic anhydride, urea, ammonium molybdate, and zinc chloride were added to nitrobenzene, were stirred and heated under reflux, and a precipitate was collected. Next, sodium hydroxide was added to the precipitate to cause hydrolysis thereof and then hydrochloric acid was added in order to provide acidic conditions and thereby yield zinc phthalocyanine tetracarboxylic acid.

Next, the zinc phthalocyanine tetracarboxylic acid and 2-aminoethanethiol (produced by Tokyo Chemical Industry Co., Ltd.) were mixed with a mass ratio of 1:2 in methanol to prepare a mixed solution. The prepared mixed solution was caused to react for 60 minutes using an ultrasonic cleaner and the resultant reaction liquid was filtered through a 3 μm PTFE filter. Filtered-off solid was washed three times with methanol and was then dried under reduced pressure to yield a phthalocyanine derivative [G] represented by the following structural formula (8) as a colored compound (dye).

### <MS Analysis Results>

M/Z=186 (phthalocyanine ion moiety)

M/Z=78 (2-aminoethanethiol ion moiety)

E1% 1 cm: 942

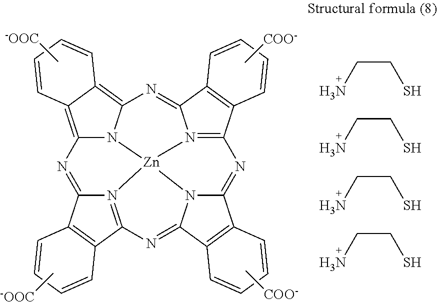

### Example 8

Alcian blue 8GX (produced by Sigma-Aldrich Co. LLC.) and sodium 1-octadecanesulfonate (produced by Tokyo Chemical Industry Co., Ltd.) were mixed with a mass ratio of 1:4 in an aqueous medium to prepare a mixed solution. The prepared mixed solution was caused to react for 60 minutes using an ultrasonic cleaner and the resultant reaction liquid was filtered through a 3 μm PTFE filter. Filtered-off solid was washed three times with water and was then dried under reduced pressure to yield a phthalocyanine derivative [H] represented by the following structural formula (9) as a colored compound (dye).

### <MS Analysis Results>

M/Z=289 (phthalocyanine ion moiety)

M/Z=333 (octadecanesulfonate ion moiety)

E1% 1 cm: 401

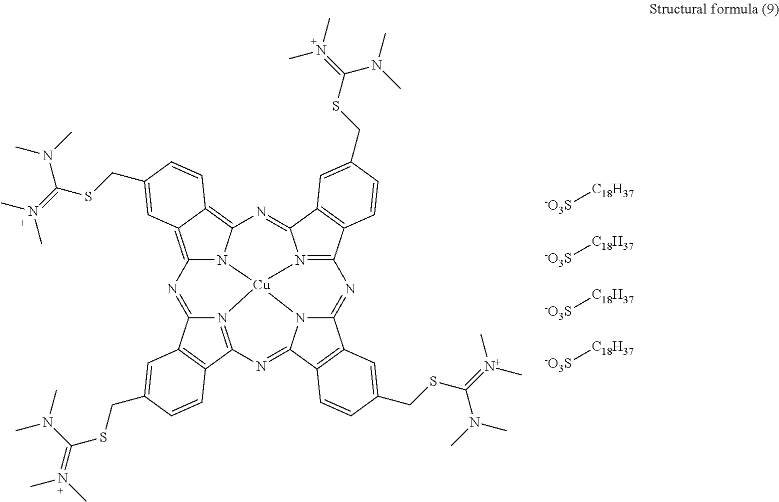

Table 1 shows evaluation results for Examples 9 to 15 in which colored compounds (dyes) according to the present disclosure were adopted in nanowire transparent conductive films as described below, and for Comparative Examples 1 to 6 provided for comparison therewith.

### Example 9

A silver nanowire dispersion liquid [1] (AgNW-25 (average diameter 25 nm, average length 23 μm) produced by Seashell Technology, LLC.) was used as metal nanowires.

A colored compound solution was prepared by the following procedure.

A solution was prepared by adding 10 mg of the phthalocyanine derivative [A] represented by structural formula (2) to 10 g of 1:1 water/ethylene glycol used as a solvent, and using an ultrasonic cleaner for 60 minutes to dissolve the phthalocyanine derivative [A]. Thereafter, the prepared solution was filtered through a PTFE filter having a pore diameter of 3 μm and the filtrate was used as the colored compound solution.

Next, 2 g of the silver nanowire dispersion liquid [1] was added to the colored compound solution and stirring was performed for 12 hours at room temperature to cause adsorption of the phthalocyanine derivative [A] represented by structural formula (2) onto the silver nanowires to obtain a dispersion liquid [2] of the silver nanowires including the adsorbed colored compound. Thereafter, the silver nanowire dispersion liquid [2] was added into a fluororesin filter paper tube No. 89 produced by Advantec MFS, Inc. and washing was performed repeatedly using 3:1 water/ethanol as a solvent until the filtrate appeared colorless and transparent to the naked eye.

The colored compound adsorbed silver nanowire dispersion liquid [2] obtained through the above process was mixed with other materials in the following formulation to prepare a dispersion liquid.

### <Formulation>

Silver nanowire dispersion liquid [2]: 0.06 mass % (net silver nanowire mass converted value)

Hydroxypropyl methylcellulose (produced by Sigma-Aldrich Co. LLC.): 0.09 mass %

Water: 89.85 mass %

Ethanol: 10 mass %

The prepared dispersion liquid coating material was coated onto a transparent substrate by a 10-count coil bar to form a dispersion film. The mass per unit area of the silver nanowires was 0.012 g/m². The transparent substrate was PET (Lumirror U34 produced by Toray Industries, Inc.) having a film thickness of 125 μm.

Next, warm air was blown onto the coated surface using a dryer with the coated surface exposed to the atmosphere in order to remove solvent from the dispersion film by drying, and drying was then performed for a further 5 minutes at 120° C. to prepare a transparent conductive film.

### Example 10

A transparent conductive film was prepared in the same way as in Example 9 with the exception that the colored compound solution was prepared using the phthalocyanine derivative [D] represented by structural formula (5) instead of the phthalocyanine derivative [A] represented by structural formula (2) that was used in Example 9. A dispersion liquid that was obtained after adsorption of the colored compound is referred to as silver nanowire dispersion liquid [3].

### Example 11

A transparent conductive film was prepared in the same way as in Example 9 with the exception that the colored compound solution was prepared using the phthalocyanine derivative [F] represented by structural formula (7) instead of the phthalocyanine derivative [A] represented by structural formula (2) that was used in Example 9. A dispersion liquid that was obtained after adsorption of the colored compound is referred to as silver nanowire dispersion liquid [4].

### Example 12

A transparent conductive film was prepared in the same way as in Example 9 with the exception that a silver nanowire dispersion liquid [5] (AW-030 (average diameter 30 nm, average length 20 μm) produced by Zhejiang Kechuang Advanced Materials Co., Ltd.) was used as the metal nanowires instead of the silver nanowire dispersion liquid [1] that was used in Example 9. A dispersion liquid that was obtained after adsorption of the colored compound is referred to as silver nanowire dispersion liquid [6].

### Example 13

A transparent conductive film was prepared in the same way as in Example 9 with the exception that a silver nanowire dispersion liquid [7] (Agnws-40 (average diameter 40 nm, average length 30 μm or more) produced by ACS Material) was used as the metal nanowires instead of the silver nanowire dispersion liquid [1] that was used in Example 9. A dispersion liquid that was obtained after adsorption of the colored compound is referred to as silver nanowire dispersion liquid [8].

### Example 14

A transparent conductive film was prepared in the same way as in Example 9 with the exception that a copper nanowire dispersion liquid [1] (NovaWireCu01 (average diameter 100 nm, average length 30 μm) produced by Novarials Corporation) was used as the metal nanowires instead of the silver nanowire dispersion liquid [1] that was used in Example 9. A dispersion liquid that was obtained after adsorption of the colored compound is referred to as copper nanowire dispersion liquid [3].

### Example 15

A transparent conductive film was prepared in the same way as in Example 9 with the exception that the colored compound solution was prepared using the phthalocyanine derivative [H] represented by structural formula (9) instead of the phthalocyanine derivative [A] represented by structural formula (2) that was used in Example 9. A dispersion liquid that was obtained after adsorption of the colored compound is referred to as silver nanowire dispersion liquid [9].

### Comparative Example 1

A transparent conductive film was prepared in the same way as in Example 9 with the exception that a silver nanowire-containing dispersion liquid coating material prepared according to the following formulation was used instead of the dispersion liquid used in Example 9. which was prepared according the specified formulation using the colored compound adsorbed silver nanowire dispersion liquid [2].

### <Formulation>

Silver nanowire dispersion liquid [1]: 0.06 mass % (net silver nanowire mass converted value)

Hydroxypropyl methylcellulose (produced by Sigma-Aldrich Co. LLC.): 0.09 mass %

Water: 89.85 mass %

Ethanol: 10 mass %

### Comparative Example 2

A transparent conductive film was prepared in the same way as in Example 9 with the exception that a silver nanowire-containing dispersion liquid coating material prepared according to the following formulation was used instead of the dispersion liquid used in Example 9, which was prepared according to the specified formulation using the colored compound adsorbed silver nanowire dispersion liquid [2].

### <Formulation>

Silver nanowire dispersion liquid [5]: 0.06 mass % (net silver nanowire mass converted value)

Hydroxypropyl methylcellulose (produced by Sigma-Aldrich Co. LLC.): 0.09 mass %

Water: 89.85 mass %

Ethanol: 10 mass %

### Comparative Example 3

A transparent conductive film was prepared in the same way as in Example 9 with the exception that a silver nanowire-containing dispersion liquid coating material prepared according to the following formulation was used instead of the dispersion liquid used in Example 9, which was prepared according to the specified formulation using the colored compound adsorbed silver nanowire dispersion liquid [2].

### <Formulation>

Silver nanowire dispersion liquid [7]: 0.06 mass % (net silver nanowire mass converted value)

Hydroxypropyl methylcellulose (produced by Sigma-Aldrich Co. LLC.): 0.09 mass %

Water: 89.85 mass %

Ethanol: 10 mass %

### Comparative Example 4

A transparent conductive film was prepared in the same way as in Example 9 with the exception that a colored compound prepared by the following procedure was used as the colored compound instead of the phthalocyanine derivative [A] represented by structural formula (2) that was used in Example 9. A dispersion liquid used in Comparative Example 4 that was obtained after adsorption of the colored compound is referred to as silver nanowire dispersion liquid [10].

The colored compound was prepared by the following procedure.

Lanyl Black BG E/C (produced by Taoka Chemical Co., Ltd.) and 2-aminoethanethiol hydrochloride (produced by Wako Pure Chemical Industries, Ltd.) were mixed with a mass ratio of 4:1 in an aqueous medium to prepare a mixed solution. The prepared mixed solution was caused to react for 100 minutes using an ultrasonic cleaner and was then left for 15 hours. The reaction liquid was filtered through a mixed cellulose ester type membrane filter having a pore diameter of 3 μm. Filtered-off solid was washed three times with water and was then dried at 100° C. in a vacuum oven to yield a chromium complex derivative [K].

A dispersion liquid that was obtained after adsorption of the colored compound is referred to as silver nanowire dispersion liquid [10].

### <MS Analysis Results>

M/Z=420 (chromium complex moiety)

M/Z=78 (2-aminoethanethiol ion moiety)

E1% 1 cm: 201

### Comparative Example 5

A transparent conductive film was prepared in the same way as in Comparative Example 4 with that exception that calendering (nip width 1 mm, load 4 kN, speed 1 m/minute) was carried out after coating and drying, whereas calendering was not carried out after coating and drying in Comparative Example 4.

### Comparative Example 6

A transparent conductive film was prepared in the same way as in Example 9 with the exception that a copper nanowire-containing dispersion liquid coating material prepared according to the following formulation was used instead of the dispersion liquid used in Example 9, which was prepared according to the specified formulation using the colored compound adsorbed silver nanowire dispersion liquid [2].

### <Formulation>

Copper nanowire dispersion liquid [1]: 0.06 mass % (net copper nanowire mass converted value)

Hydroxypropyl methylcellulose (produced by Sigma-Aldrich Co. LLC.): 0.09 mass %

Water: 89.85 mass %

Ethanol: 10 mass %

(Evaluation)

The transparent conductive films prepared in Examples 9 to 15 and Comparative Examples 1 to 6 described above were evaluated in terms of A) total light transmittivity [%], B) haze value, C) sheet resistance value [Ω/sq.], D) Δreflection L* value, E) change in sheet resistance in an environment test at 60° C. and 90% humidity, and F) change in sheet resistance after an environment test under Xe lamp irradiation. These evaluations were carried out as follows.

### (A) Evaluation of Total Light Transmittivity

An HM-150 (product name; produced by Murakami Color Research Laboratory Co., Ltd.) was used to evaluate the total light transmittivity of each of the transparent conductive films in accordance with JIS K7136.

### (B) Evaluation of Haze Value

An HM-150 (product name; produced by Murakami Color Research Laboratory Co., Ltd.) was used to evaluate a haze value of each of the transparent conductive films in accordance with JIS K7136. Note that a smaller haze value is more preferable.

### (C) Evaluation of Sheet Resistance Value

An EC-80P (product name; produced by Napson Corporation) was used to evaluate a sheet resistance value of each of the transparent conductive films. A sheet resistance value of 200 [Ω/sq.] or less is preferable.

### (D) Evaluation of ΔReflection L* Value

The Δreflection L* value was evaluated by attaching black plastic tape (VT-50 produced by Nichiban Co., Ltd.) at the metal nanowire layer-side of the transparent conductive film and performing evaluation from the opposite side to the metal nanowire layer-side in accordance with JIS Z8722 using a Color i5 produced by X-Rite Inc. The light source was a D65 light source and an average value of measurements performed at three arbitrary locations by an SCE (specular component excluded) method was taken to be a reflection L value. Herein, the Δreflection L* value can be calculated using the following formula.

ΔReflection L*value=(Reflection L*value of transparent electrode including substrate)−(Reflection L*value of substrate)

Note that a smaller Δreflection L* value is more preferable.

### (E) Evaluation of Change in Sheet Resistance in Environment Test at 60° C. and 90% Humidity

The PET film and a glass slide (product no.: S9213, produced by Matsunami Glass Ind., Ltd.) were affixed to each other by using adhesive film (product no.: 8146-2, produced by 3M) on the glass slide such that the metal nanowire layer surface was positioned against the glass.

Next, the resultant product was placed in a glass slide holder and was left for 500 hours in an oven set to a temperature of 60° C. and a humidity of 90%. Thereafter, the sheet resistance value was evaluated.

Evaluation results were compared for corresponding metal nanowires having a colored compound adsorbed thereon and not having a colored compound adsorbed thereon.

### <Evaluation Standard>

Good: Smaller rate of change when colored compound adsorbed than when not adsorbed

Poor: Larger rate of change when colored compound adsorbed than when not adsorbed

(F) Evaluation of Change in Sheet Resistance after Environment Test Under Xe Lamp Irradiation

The PET film and a glass slide (product no.: S9213, produced by Matsunami Glass Ind., Ltd.) were affixed to each other using adhesive film (product no.: 8146-2, produced by 3M) on the glass slide such that the metal nanowire layer surface was positioned against the glass.

Next, the resultant product was placed in a glass slide holder and was subjected to a Xe lamp light resistance test for 100 hours. Thereafter, the sheet resistance value was evaluated.

Evaluation results were compared for corresponding metal nanowires having a colored compound adsorbed thereon and not having a colored compound adsorbed thereon.

### <Evaluation Standard>

Good: Smaller rate of change when colored compound adsorbed than when not adsorbed

Poor: Larger rate of change when colored compound adsorbed than when not adsorbed

TABLE 1

| | | Metal nanowires | | | | Colored |
|---|---|---|---|---|---|---|
| | Metal nanowires | with adsorbed | Colored | El % | Solubility of colored | compound |
| | used | colored compound | compound | 1 cm | compound in water | particle diameter |
| Example 9 | Silver nanowire | Silver nanowire | [A] | 581 | 0.03 mass % < | <3 μm |
| | dispersion liquid [1] | dispersion liquid [2] | | | | |
| Example 10 | Silver nanowire | Silver nanowire | [D] | 692 | 0.03 mass % < | <3 μm |
| | dispersion liquid [1] | dispersion liquid [3] | | | | |
| Example 11 | Silver nanowire | Silver nanowire | [F] | 875 | 0.03 mass % < | <3 μm |
| | dispersion liquid [1] | dispersion liquid [4] | | | | |
| Example 12 | Silver nanowire | Silver nanowire | [A] | 581 | 0.03 mass % < | <3 μm |
| | dispersion liquid [5] | dispersion liquid [6] | | | | |
| Example 13 | Silver nanowire | Silver nanowire | [A] | 581 | 0.03 mass % < | <3 μm |
| | dispersion liquid [7] | dispersion liquid [8] | | | | |
| Example 14 | Copper nanowire | Copper nanowire | [A] | 581 | 0.03 mass % < | <3 μm |
| | dispersion liquid [1] | dispersion liquid [3] | | | | |
| Example 15 | Silver nanowire | Silver nanowire | [H] | 401 | 0.03 mass % < | <3 μm |
| | dispersion liquid [1] | dispersion liquid [9] | | | | |
| Comparative | Silver nanowire | — | — | — | — | — |
| Example 1 | dispersion liquid [1] | | | | | |
| Comparative | Silver nanowire | — | — | — | — | — |
| Example 2 | dispersion liquid [5] | | | | | |
| Comparative | Silver nanowire | — | — | — | — | — |
| Example 3 | dispersion liquid [7] | | | | | |
| Comparative | Silver nanowire | Silver nanowire | [K] | 201 | 0.03 mass % < | <3 μm |
| Example 4 | dispersion liquid [1] | dispersion liquid [10] | | | | |
| Comparative | Silver nanowire | Silver nanowire | [K] | 201 | 0.03 mass % < | <3 μm |
| Example 5 | dispersion liquid [1] | dispersion liquid [10] | | | | |
| Comparative | Copper nanowire | — | — | — | — | — |
| Example 6 | dispersion liquid [1] | | | | | |

| | pH of colored | | | | C) | | E) | |
|---|---|---|---|---|---|---|---|---|
| | compound | | A) | B) | Sheet | D) | After 500 hr | F) |
| | aqueous | Pressing | Total light | Haze | resistance | ΔReflection | at 60° C. and | 100 hr Xe light |
| | solution | treatment | transmittivity | value | value | L* value | 90% humidity | resistance test |
| Example 9 | 6.8 | No | 92.0 | 0.9 | 100 | 1.7 | Good | Good |
| Example 10 | 6.8 | No | 92.0 | 0.9 | 100 | 1.7 | Good | Good |
| Example 11 | 4.8 | No | 91.9 | 0.9 | 100 | 1.7 | Good | Good |
| Example 12 | 6.8 | No | 91.7 | 1.0 | 100 | 2.0 | Good | Good |
| Example 13 | 6.8 | No | 91.5 | 1.5 | 100 | 3.2 | Good | Good |
| Example 14 | 6.8 | No | 88.5 | 2.0 | 100 | 4.0 | Good | Good |
| Example 15 | 6.8 | No | 91.9 | 0.9 | 100 | 1.8 | Good | Good |
| Comparative | — | No | 91.6 | 1.1 | 100 | 2.5 | Poor | Good |
| Example 1 | | | | | | | | |
| Comparative | — | No | 91.4 | 1.2 | 100 | 2.9 | Poor | Good |
| Example 2 | | | | | | | | |
| Comparative | — | No | 91.0 | 2.0 | 100 | 4.1 | Poor | Good |
| Example 3 | | | | | | | | |
| Comparative | 5.5 | No | 91.9 | 1.0 | 300 | 1.9 | Good | Good |
| Example 4 | | | | | | | | |
| Comparative | 5.5 | Yes | 91.9 | 1.0 | 100 | 1.9 | Good | Good |
| Example 5 | | | | | | | | |
| Comparative | — | No | 88.0 | 3.3 | 100 | 5.0 | Poor | Good |
| Example 6 | | | | | | | | |

### INDUSTRIAL APPLICABILITY

The phthalocyanine-based complex compound according to the present disclosure is particularly suitable for use in a transparent conductive film of a touch panel, but is also suitable for applications other than transparent conductive films of touch panels (for example, organic EL electrodes, solar cell surface electrodes, transparent antennas (wireless antennas for charging in mobile telephones and smartphones), and transparent heaters that can be used for condensation prevention or the like).

### REFERENCE SIGNS LIST

6 metal nanowire bodies
7 colored compound (dye)
8 binder layer
9 substrate
10 overcoating layer
11 anchor layer

1. A phthalocyanine-based complex compound represented by general formula (1) shown below, 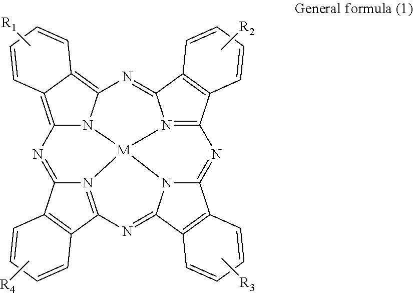
where M in general formula (1) is any of Cu, Fe, Ti, V, Ni, Pd, Pt, Pb, Si, Bi, Cd, La, Tb, Ce, Be, Mg, Co, Ru, Mn, Cr, Mo, Sn, and Zn, and may be present or absent,
one or more of R₁ to R₄ in general formula (1) are present on a phthalocyanine moiety, each include an ion represented by any general formula in general formula group (A) shown below, and may be the same or different to one another, 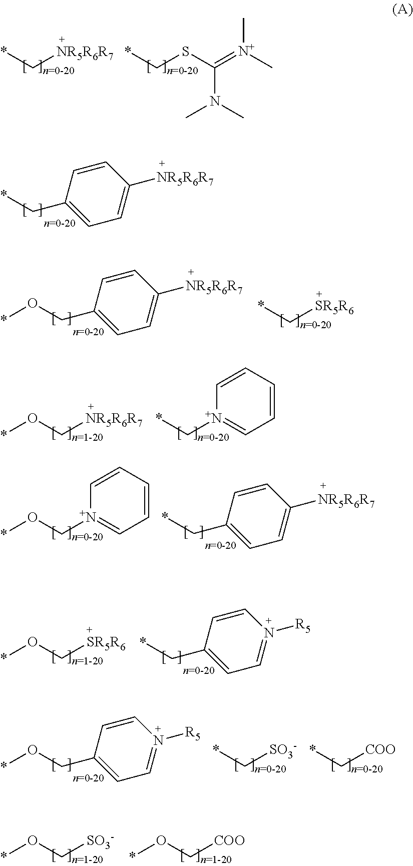
R₅ to R₇ in general formula group (A) are each hydrogen or a hydrocarbon group, and may be the same or different to one another,
R₁ to R₄ in general formula (1) each further include a counter ion represented by any general formula in general formula group (B) shown below, 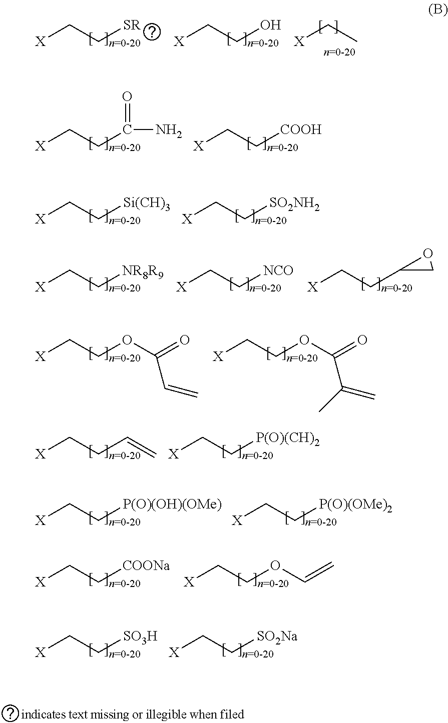
X in general formula group (B) is an ion represented by SO₃⁻, COO⁻, PO₃H⁻, PO₃²⁻, N⁺R₈R₉R₁₀, or PhN⁺R₈R₉R₁₀, an ion represented by general formula (2) shown below, or an ion represented by structural formula (1) shown below, 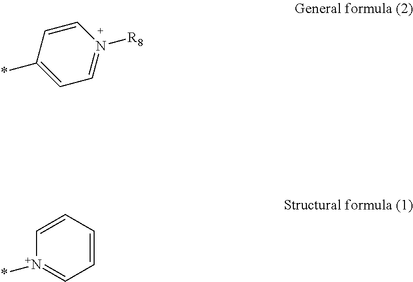
and R₈ to R₁₀ in general formula group (B), N⁺R₈R₉R₁₀, PhN⁺R₈R₉R₁₀, and general formula (2) are each hydrogen or a hydrocarbon group, and may be the same or different to one another.

2. The phthalocyanine-based complex compound of claim 1 having an E1% 1 cm value of at least 300 at a wavelength of maximum absorption in a visible light region.

3. The phthalocyanine-based complex compound of claim 1, wherein
the phthalocyanine-based complex compound dissolves in water or ethylene glycol in an amount of at least 0.01 mass %.

4. The phthalocyanine-based complex compound of claim 1, wherein
the phthalocyanine-based complex compound dissolves as molecules or disperses as particles having a number average particle diameter of no greater than 3 μm in water or ethylene glycol.

5. The phthalocyanine-based complex compound of claim 1, wherein
the phthalocyanine-based complex compound forms a solution having a hydrogen ion concentration (pH) of 4 to 10 when dissolved in water in an amount of 0.1 mass %.

6. A method of producing a phthalocyanine-based complex compound, for use in producing the phthalocyanine-based complex compound of claim 1, comprising
preparing a raw material solution in which a raw material including a phthalocyanine derivative moiety is dissolved in a solvent and a compound solution in which a compound including a moiety that adsorbs onto a metal is dissolved in a solvent, and mixing the raw material solution and the compound solution to precipitate a phthalocyanine-based complex compound.

7. The phthalocyanine-based complex compound of claim 2, wherein
the phthalocyanine-based complex compound dissolves in water or ethylene glycol in an amount of at least 0.01 mass %.

8. The phthalocyanine-based complex compound of claim 2, wherein
the phthalocyanine-based complex compound dissolves as molecules or disperses as particles having a number average particle diameter of no greater than 3 μm in water or ethylene glycol.

9. The phthalocyanine-based complex compound of claim 2, wherein
the phthalocyanine-based complex compound forms a solution having a hydrogen ion concentration (pH) of 4 to 10 when dissolved in water in an amount of 0.1 mass %.

10. A method of producing a phthalocyanine-based complex compound, for use in producing the phthalocyanine-based complex compound of claim 2, comprising
preparing a raw material solution in which a raw material including a phthalocyanine derivative moiety is dissolved in a solvent and a compound solution in which a compound including a moiety that adsorbs onto a metal is dissolved in a solvent, and mixing the raw material solution and the compound solution to precipitate a phthalocyanine-based complex compound.
